(19) United States
(12) Patent Application Publication
(10) Pub. No.: US 2014/0066768 A1
(43) Pub. Date: Mar. 6, 2014

(54) Frequency Distribution in Harmonic Ultrasound Imaging

(71) Applicants: Lei Sui, Newcastle, WA (US); Rushabh Modi, Issaquah, WA (US)

(72) Inventors: Lei Sui, Newcastle, WA (US); Rushabh Modi, Issaquah, WA (US)

(73) Assignee: Siemens Medical Solutions USA, Inc., Malvern, PA (US)

(21) Appl. No.: 13/599,585

(22) Filed: Aug. 30, 2012

(51) Int. Cl. A61B 8/13

(52) U.S. Cl. 600/443

(57) ABSTRACT

Frequency variation is used in frequency compounding. A phase inversion harmonic image is compounded with a downshift harmonic image. The depths for downshifting fractional harmonics are determined based on a signal-to-noise ratio of the harmonic information at a given harmonic. The depth for transition between one type of harmonic imaging (e.g., phase inversion) and another (e.g., downshifted harmonic) is determined based on a similarity of the one type with noise. Weights used for frequency compounding are determined based on a difference between noise and one of the types of data to be compounded, and spatially steering angles.

### BACKGROUND

The present embodiments relate to harmonic ultrasound imaging. In particular, frequency variation is provided in harmonic ultrasound imaging.

For harmonic imaging, acoustic energy is transmitted at a fundamental frequency, such as 2 MHz. Response of tissue or other structure to the acoustic energy is measured as a harmonic frequency, such as a 4 MHz second harmonic (i.e., second order of the fundamental frequency).

Various approaches may be used to isolate information at the desired harmonic frequency or frequency band. For example, two pulses are transmitted 180 degrees out of phase. By summing the responses, the information at the odd harmonic frequency, including the fundamental frequency, cancels and the information at the even harmonic frequency, including the second harmonic, is maintained. FIG. 8 shows an example harmonic image obtained with this pulse inversion approach. Higher frequencies have less penetration depth than lower frequencies. FIG. 8 shows the tissue response decreasing with depth.

To increase penetration depth for harmonic imaging, different frequencies may be used. Rather than phase inversion, the received signals may be filtered with a band pass filter. For deeper depths, the band pass is downshifted to lesser frequencies, such as downshifting in increments of 0.1 MHz from 4 MHz second harmonic to a 3 MHz 1.5 fractional harmonic. This downshifting of the filtering may improve penetration, but results in a relatively coarse image appearance. FIG. 9 shows this improved depth, but with a seemingly lesser resolution.

In another approach, penetration depth is increased by combining the phase inversion harmonic information with response at the fundamental frequency. Frequency compounding uses the harmonic information for shallow depths and uses the fundamental information for greater depths. FIG. 10 shows an example. The image has a harmonic imaging look and feel at shallow depth, but a different look and feel for deeper depths.

### BRIEF SUMMARY

By way of introduction, the preferred embodiments described below include methods, computer readable media, instructions, and systems for ultrasound imaging with frequency variation. In one approach, the depths for downshifting fractional harmonics are determined, such as determining based on a signal-to-noise ratio of the harmonic information at a given harmonic. In another approach, the depth for transition between one type of harmonic imaging (e.g., phase inversion) and another (e.g., downshifted harmonic) is determined, such as based on a similarity of the one type with noise. In yet another approach, weights used for frequency compounding are adaptively determined, based on a difference between noise and one of the types of data to be compounded. These approaches may be used separately or in any combination of two or all of the approaches.

In a first aspect, a method is provided for frequency distribution in harmonic ultrasound imaging. Using transmissions at different phases, a first frame of ultrasound data representing response of a region of a patient at one harmonic frequency is generated. Using filtering at different pass bands, a second frame of ultrasound data at a plurality of different harmonic frequencies as a function of depth is generated. The ultrasound data of the first frame is combined with the ultrasound data of the second frame into a third frame. A relative contribution of the ultrasound data of the first frame to the ultrasound data of the second frame is a function of depth such that the contribution in a near field is greater from the first frame and the contribution in a far field is greater from the second frame. An image of the region is generated with the combined ultrasound data of the third frame.

In a second aspect, a non-transitory computer readable storage medium has stored therein data representing instructions executable by a programmed processor for frequency distribution in harmonic ultrasound imaging. The storage medium includes instructions for detecting a first penetration depth of harmonic imaging at a first harmonic frequency, shifting to a second harmonic frequency lower than the first harmonic frequency at the first penetration depth, detecting a second penetration depth of the harmonic imaging at the second harmonic frequency, and shifting to a third harmonic frequency lower than the first harmonic frequency at the second penetration depth.

In a third aspect, a non-transitory computer readable storage medium has stored therein data representing instructions executable by a programmed processor for frequency distribution in harmonic ultrasound imaging. The storage medium includes instructions for acquiring first and second sets of harmonic data, the first set of harmonic data being different from the second set of harmonic data, compounding the harmonic data of the first set with the harmonic data of the second set, the compounding being a weighted average, and calculating weights of the weighted average as a function of a difference of the harmonic data of the first set with noise.

Further aspects and advantages of the invention are discussed below in conjunction with the preferred embodiments. The present invention is defined by the following claims, and nothing in this section should be taken as a limitation on those claims.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 is a flow chart diagram of one embodiment of a method for frequency distribution in harmonic ultrasound imaging using three approaches;

FIG. 2 is a flow chart diagram of one embodiment of a method for frequency distribution in harmonic ultrasound imaging using downshift;

FIG. 3 illustrates different sub-portions of a field of view;

FIG. 4 is a flow chart diagram of one embodiment of a method for frequency distribution in harmonic ultrasound imaging using combination of different types of harmonic imaging;

FIG. 5 is a flow chart diagram of one embodiment of a method for frequency distribution in harmonic ultrasound imaging using frequency compounding with adaptive weights;

FIG. 6 is a medical diagnostic ultrasound harmonic image generated using the method of FIG. 1 (using the approaches of FIGS. 2, 4, and 5);

FIG. 7 is a block diagram representing one embodiment of a system for frequency distribution in harmonic ultrasound imaging;

FIG. 8 is a prior art medical diagnostic ultrasound harmonic image generated using phase inversion;

FIG. 9 is a prior art medical diagnostic ultrasound harmonic image generated using frequency downshift; and

FIG. 10 is a prior art medical diagnostic ultrasound harmonic image generated using frequency compounding.

### DETAILED DESCRIPTION

Dynamic downshifted harmonic imaging is generated. The depths for downshifting may be determined dynamically, such as based on comparison to noise. The dynamic downshift information may be modulated with another type of harmonic information, such as phase inversion information. The depth for transition in the combination may be based on a comparison of noise to the other type of harmonic information. The weighting for frequency compounding in the combination may be determined as a function of the downshift frequency after the downshift image is modified by phase shift harmonic data characteristics. Image appearance may be improved while maintaining penetration depth.

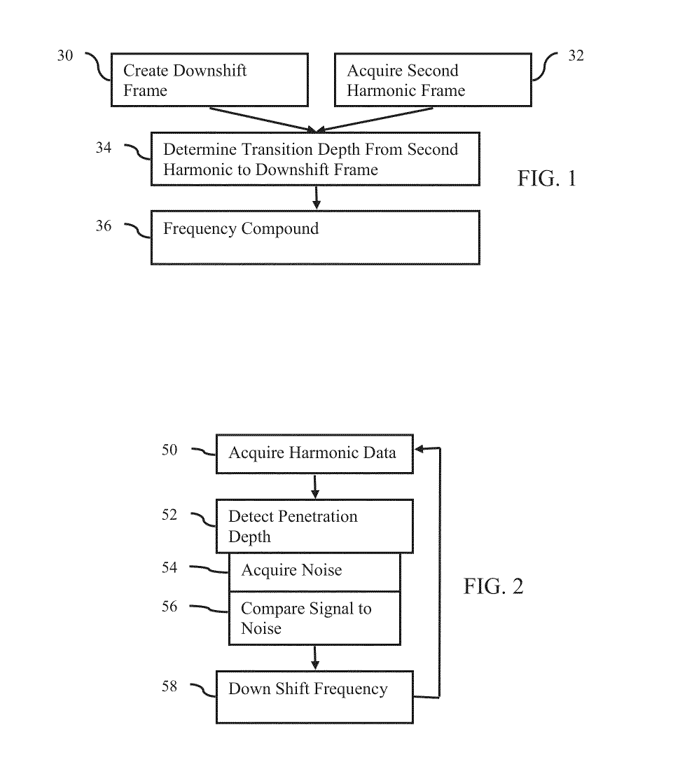

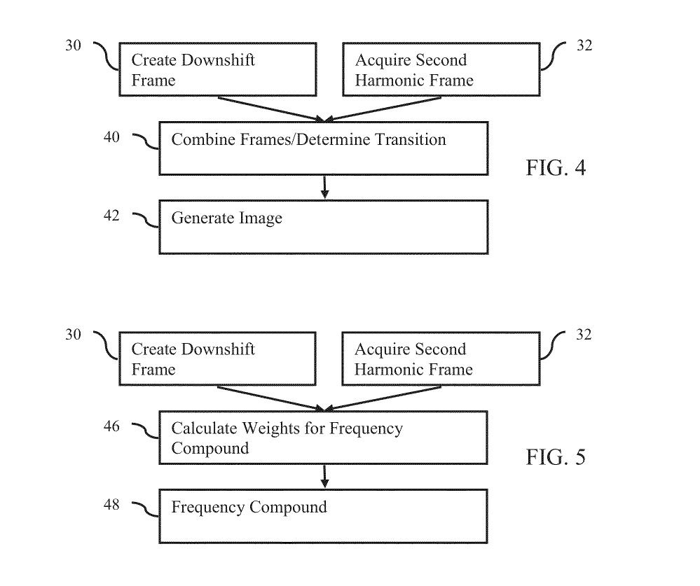

For example, the approach of FIG. 2 is used to decide at which depth the higher frequency loses penetration and a lower frequency has to take over. This is done by comparing the signal frame with noise frame at that depth. If the loss of penetration is detected and a lower frequency may improve penetration, the approach of FIG. 4 is used to control the system to do frequency compounding. If the frequency compounding is warranted in the approach of FIG. 4, the 2^{nd} harmonic weights of compounding at different depths are computed in the approach of FIG. 5. The weights are proportional to the signal to noise ratio of the 2^{nd} harmonic.

The weights between frequencies and/or breakpoints between downshifts may be automatically calibrated as a function of the difference between the signal-to-noise ratios of the downshift or harmonic information. The difference of signal measurements between with downshifts or harmonics and without is used. The compounding weights are estimated based on signal-to-noise ratio of the compounding sources with or without the knowledge about noise characteristics. Speckle patterns (size, variance) in a region without downshifting may be estimated and added to a region for downshifting to mimic the patterns.

In one embodiment, the frequency distribution approaches are used with steered spatial compounding. The harmonic frames, such as from downshifting, are acquired at different steering angles. The frames from different steering angles are compounded. The frequency and steered spatial compounding may be performed together, such as weighted averaging from component frames at different frequencies and steering angles. When combined with steered spatial compounding, the frequency compounding weights are further a function of steering angles. Due to the difference in steering between component frames, different numbers of frames are available for compounding at different locations. The weights adjust to the number of frames due to the spatial steering.

FIG. 1 shows a method for frequency distribution in harmonic ultrasound imaging. The method incorporates three approaches. In particular, the method includes generation of a downshift frame of data in act 30. FIG. 2 provides one embodiment of act 30. The method of FIG. 1 incorporates the determination of the transition depth or break point for combining two different types of harmonic information. For example, filtering is used to generate a downshift image. Phase inversion is used to create a second harmonic image. The depth breakpoint at which the downshifted information is added to or replaces the phase inversion information is determined based on the depth of penetration. FIG. 4 provides one embodiment of act 34. The method of FIG. 1 also incorporates frequency compounding in act 36. The relative contribution of the different frames or different types of harmonic information at any depth may be determined based on deviation of the harmonic information from noise. FIG. 5 provides one embodiment of act 36.

In alternative embodiments, any of acts 30, 34, or 36 are used alone or without the other acts. In other embodiments, combinations of two of the acts are used.

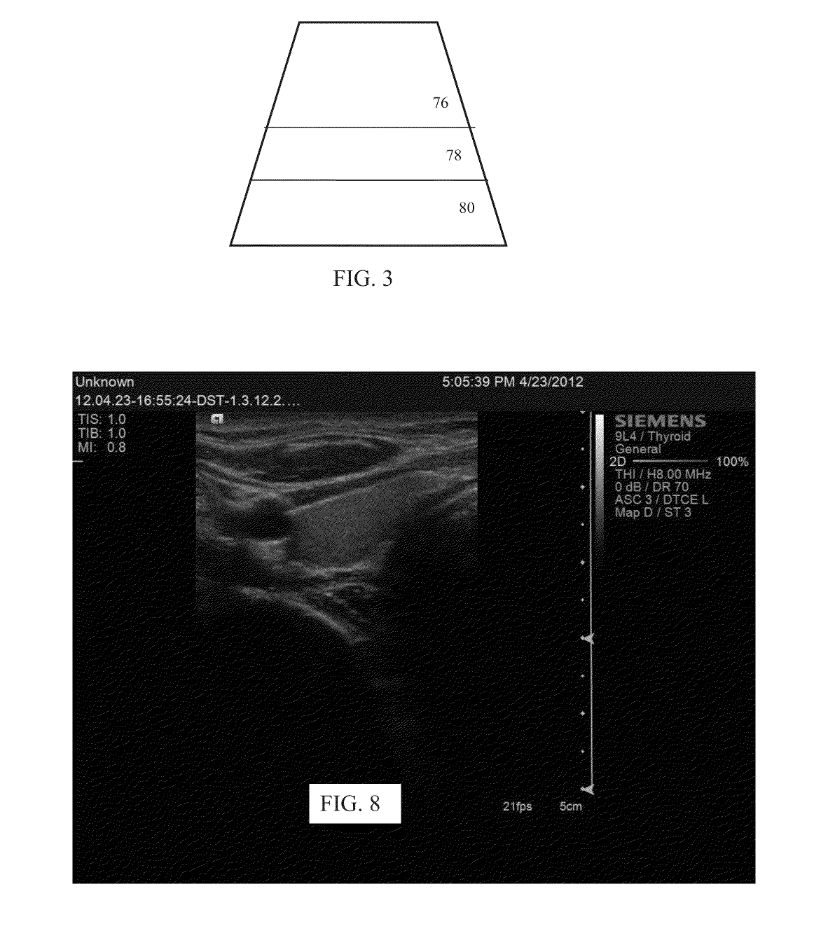

In act 30, a frame of downshifted frequencies is generated. Different frequencies are used at different depths. Any number of depth bands may be used, such as dividing the desired field of view or depth into two or more bands. FIG. 3 shows three bands 76, 78, 80 associated with different depths. Another example is eight bands. Each sampling depth may be part of a band with other depths. Alternatively, each depth is treated as one band. The bands are equally distributed over depth or may have different ranges of depths.

All of the frequencies are harmonic frequencies. For example, the transmit frequency is 2 MHz. The near field band 76 is at the second harmonic (i.e., second order of the fundamental frequency). The other bands are at fractional harmonics greater than the fundamental frequency, such as being at 2.1 MHz or greater. Any step size in frequency transition may be used, such as downshifting from 4 MHz to 3 MHz in 0.1 MHz steps. Alternatively, one of the frequencies used is the fundamental frequency.

The downshifting is by changing a filtering pass band. While the singular frequency is used herein, the operation is for a band of frequencies centered on the frequency. For example, the second harmonic at 4 MHz may include signals at other frequencies, such as a 3.8-4.2 MHz band. The filtering isolates information in the pass band. Using filtering, information at frequencies outside this pass band may still be in the signal, but at a reduced amplitude.

The information at harmonic frequencies is obtained by filtering. Acoustic energy is transmitted. The acoustic energy is generated from a pulse of one or more cycles. The frequency of the cycles of the pulse or signal is the fundamental frequency. Echo signals are generated in response to the acoustic energy. The echoes may have a broadband characteristic, such as including information at the fundamental frequency, second harmonic frequency and other harmonic frequencies. The received signals may be filtered to isolate information at a desired pass band, such as by band pass filtering or a sequential combination of high and low pass filtering. In other embodiments, the signals are downshifted by a programmable amount and low pass filtered to isolate information at the desired pass band.

The downshift of the pass band may be predetermined. For example, the penetration of the harmonic signals at different frequencies is assumed. The frequency is downshifted to likely provide a highest harmonic that is sufficiently above a noise level for any given depth. Any number of depth regions or bands and corresponding number of different downshifted frequencies and frequency step sizes may be used.

In an alternative embodiment shown in FIG. 2, the downshifted frame of data is acquired with the downshift established dynamically or adaptively. FIG. 2 shows an approach for automatic calibration of the breakpoints (i.e., depths) of frequency downshift. Additional, different, or fewer acts may be provided. For example, the penetration depth is determined without acquisition of noise in act 54 and/or comparison in act 56.

In act 50, harmonic data is acquired. The data is acquired by transmission at the fundamental frequency and reception at the harmonic frequency. In one embodiment, one of the transmissions and receptions used for phase inversion is also used for acquiring the downshift frame. Phase inversion uses two or more transmissions and receptions for a given location. The received signals from one of these transmissions may be used to generate the downshift frame of ultrasound harmonic data. In other embodiments, separate acquisition is performed.

For an initial iteration, the data is acquired by filtering at a frequency desired for the image. For example, the initial iteration acquires ultrasound data at the second harmonic or one downshifted step from the second harmonic.

For later iterations, the ultrasound data is acquired at the downshifted frequency. Using programmed, user selected, or automatically tested step sizes, the harmonic data is acquired at a frequency one step down from a previous iteration. Where a lack of penetration is detected in act 52, the frequency is downshifted in act 58 and harmonic data is acquired at the downshifted frequency in act 50.

In act 52, the penetration depth of the current harmonic is detected. The data received at the current harmonic in act 50 is used to determine the penetration depth in act 52. For example, in an initial iteration, the penetration depth of a second harmonic obtained by filtering, phase inversion, or other technique is determined. For later iterations after downshifting, the penetration depth of the downshifted harmonic obtained by filtering is determined.

Intensity of the response at the harmonic frequency may be used to determine penetration depth. The intensity may be a single value or a combination of values. For example, the intensities at a given depth and/or over a range of depths are averaged. The penetration along one scan line, along multiple scan lines, or for the field of view is determined. The intensity is determined for each depth or range of depths. In one embodiment, the field of view is divided into a plurality of bands (see FIG. 3). For example, eight bands are used. The intensity for the depth at the bottom of each band is calculated. To account for fluid regions, the values for locations associated with fluid may not be included.

In one embodiment, the penetration depth of the current harmonic is detected using a signal-to-noise ratio (SNR). In act 54, noise data is acquired. Reception is performed without transmission. The transmitters are turned off and data is received. The data represents system noise and/or noise from the scanned region. Other techniques for measuring noise may be used. The noise is measured for the entire field of view, for one or more bands and not others, for some depths and not others, and/or for one or more scan lines and not others.

In act 56, the signal is compared to the noise. The signal may be the intensity. For example, the intensities and noise are histogrammed. The values for a depth, in a band, or along a scan line are plotted to a histogram. One or more statistics are calculated from the histograms to separate the noise from the signal. The statistic may be between the histograms or statistics are calculated for each histogram and compared. Any statistic may be used, such as variance. Where a threshold level of similarity occurs, the signal is considered noise and has not penetrated.

In other embodiments, a statistic of the harmonic data is compared to a statistic of the noise data. The statistic is calculated for values for a depth, in a band, or along a scan line. For example, a mean and variance of the harmonic data in a band is compared with a mean and variance of the noise data in the band. Other statistics may be used alone or in combination. Where a threshold level of similarity occurs for the statistic or statistics, the signal is considered noise and has not penetrated.

Other comparisons or penetration depth detection may be used. For example, the SNR may be calculated and compared to a threshold to determine penetration depth.

In another embodiment, penetration may be determined by comparing the intensity to a threshold. The first depth or band at which the intensity is below the threshold indicates little or no penetration. The next shallowest depth or band associated with intensity above the threshold is the penetration depth.

The comparison separates the noise from the signal. The separation identifies the deepest depth or band of depths which includes signal rather than noise. This depth or band indicates the penetration of the current harmonic.

To extend the depth beyond the penetration depth, the frequency is downshifted in act 58. The reception and corresponding filtering are shifted to isolate information in a desired pass band. For depth beyond the penetration, the acquisition is downshifted so that the harmonic data has tissue signal in deeper depths. Different frequencies are used for the different depths or bands.

Any amount of downshift may be used. In one embodiment, the downshift is performed in increments of 0.1 MHz, but greater or larger increments may be used. For the various iterations, the increments are linear. Alternatively, a non-linear function may be used for the downshifting increments. In yet another embodiment, the amount of downshift is based on the level of similarity in the comparison of noise with the information at the harmonic. Greater similarity is mapped to a larger increment.

For each downshift, the penetration depth is deeper. Lower frequency acoustic energy propagates further. By shifting the harmonic frequency to be lower, greater penetration results. The reception frequency is downshifted while keeping the transmission frequency. In one embodiment, the acquisition is the same, such as receiving for the entire field of view. The data is filtered with different pass bands for different depths or bands without repeating acquisition. For example, ultrasound data is acquired for phase inversion-based second harmonic imaging. The signals from one of the phases may be used to generate the downshift frame of ultrasound data, at least for depths to which the second harmonic does not penetrate sufficiently. The iterations are of processing the previously acquired data so that the appropriate downshift frequencies are used for the desired band or depths. Alternatively, acquisition is repeated for each iteration.

The downshift may be by band. A highest frequency able to penetrate the depth or band is selected. For each band, a different downshift frequency is determined. Where penetration is sufficient, some bands may have a same frequency. In another embodiment using only two bands, the highest frequency that penetrates to the depth of the field of view is found and used with the second harmonic or other frequency. Intervening frequencies are not used. In yet other embodiments, the determined downshifts are mapped to a continuous function for depth. The pass band for each depth is interpolated from the sampled down shifts or derived from the continuous function such that no bands are provided or the bands have a single depth range.

Once the downshifted frequency for penetrating to the deepest depth of the field of view is determined, the harmonic frequencies for the downshift frame are set. The filtered signals for the different depths are a frame of data. The frame of data is a downshifted frame of data. The detecting and shifting of acts 52 and 58 provide two or more regions with different harmonic frequencies.

The different harmonic frequencies are used in filtering to obtain the data of the frame representing the entire field of view. The downshift frame of data represents the entire field of view with data obtained by filtering (e.g., the near field band is filtered at the second harmonic). Alternatively, the downshift frame of data is harmonic data for less than the entire field of view, such as being downshift data only for bands beyond the penetration of the phase inversion harmonic. In other embodiments, the downshift frame of data includes both the phase inversion data for the near field and the downshift filtered data for the far field.

The downshifted frame of data may be used for imaging. For example, the harmonic data of the frame is mapped to display values and displayed. Using either predetermined downshifting or dynamically determined downshifting, penetration of the entire field of view with resolution associated with harmonic frequencies results.

In other embodiments, the downshifted frame of data is used for frequency compounding as represented in acts 34 and 36 of FIG. 1. FIG. 4 shows one embodiment of determining a transition depth in act 34 of FIG. 1. The transition depth represents a location at which one type of harmonic imaging is used instead of another. For example, a near field uses phase inversion and the far field used downshift harmonic imaging. Other combinations of types of harmonic imaging may be used. The transition represents the depth at which the switch between types of harmonic imaging processes occurs.

Where the downshift frame created in act 30 (see FIG. 2) initially determines the penetration depth of one type before transitioning to downshift harmonic imaging, the transition depth is determined in the process of creating the downshift frame of data. Alternatively, a different approach is used to determine the transition, so a different depth results than did from generating the downshift frame of data. In other embodiments, the downshift frame of data is for some or all depths using filtering and does not include data from a different type of harmonic imaging.

The point of transition identifies the sources of data to be used for a common frame of data. The common frame of data is assembled from different sources or types of harmonic processing. Alternatively, the point of transition defines a location at which one type of data is to be blended with or frequency compounded with another type of data, such as using phase inversion harmonic data for a near field and compounding the phase inversion harmonic data with downshifted data for the far field. The transition defines a depth for selecting the type of data to be used or to define a depth for graduated shift between two sources of harmonic data.

The transition depth is determined automatically. Rather than rely on user selection or a predetermined depth, the ultrasound data is processed to find the switch between two types of processing or harmonic data.

In act 32, a frame of ultrasound data at a harmonic frequency is acquired. The response of the patient in a region of the field of view or part of the region is generated. The harmonic frequency may be any, such as the second harmonic, fractional harmonic greater than the fundamental, third harmonic, or other integer harmonic. The response of tissue or tissue harmonic imaging is used.

Any type of harmonic imaging may be used. In one embodiment, multiple pulses at different phases are used. Phase inversion with two pulses may be used to acquire data at even harmonics. Three or more pulses with different phases for at least two pulses and with or without amplitude modulation may be used. By combining the received signals for the same location, information at a desired harmonic or group of harmonics may be obtained. Alternatively, filtering may be used to relatively emphasize, isolate, or acquire data at the harmonic frequency. Other harmonic imaging may be used.

The acquired data at the harmonic is a frame of data. The frame represents part or all of the field of view. For example, a phase shift frame of data is acquired using phase inversion harmonic imaging. The phase shift frame of data represents an entire field of view. Since the second harmonic may not penetrate the entire field of view, some of the data of the frame may be associated more with noise than tissue response. Some data may not have any signal.

In act 32, a frame of data at one or more harmonics is acquired. The frame is acquired using a different process or type of harmonic imaging. For example, a downshift frame is acquired in act 32 and a phase inversion frame is acquired in act 30. In other embodiments, the same type of processing or harmonic imaging is used, but with different settings. For example, phase shifting is used, but to isolate information at a different frequency. As another example, downshift imaging is used, but with different downshift increments and/or starting frequencies.

In one embodiment, the frame of data acquired in act 32 is a downshift frame of data. The downshift frame of data is acquired using predetermined or adaptive downshifts. For example, the downshift frame of data is acquired by calculating the depths for the downshifting as a function of a comparison of the ultrasound data at a current pass band to noise.

In act 40, the ultrasound data of the first frame is combined with the ultrasound data of the second frame. The combination forms a third frame. The third frame represents the field of view. The combination is by selection. For any given location, the data of one of the two frames is used. The selection may be based on depth, such as using one type of data for a near field and another type for a far field. The third frame is assembled from the other frames. In another embodiment, the combination is part of frequency compounding, such as averaging or weighted averaging of the data from the different frames for a band, other portion, or all of the field of view and corresponding third frame.

For example, the phase shift frame of data is combined with the downshifted frame of data. A transition point defines a switch over of relative contribution. In one case, the transition point is a depth at which data from the downshifted frame is used instead of the phase shift frame. In another case, the transition is a point at which equal weighting is used in compounding. Above the transition depth, the phase shift frame predominates or is more heavily weighted than the downshift frame of data. Below the transition depth, the downshift frame predominates or is more heavily weighted than the phase shift frame of data. The relative contribution of the ultrasound data of the phase shift frame to the ultrasound data of the downshift frame is a function of depth.

In one embodiment, more than one transition is determined. For example, a middle band or field is formed from a combination of both frames. A near field band is formed from just one frame, such as the phase shift frame. A far field is formed from just the other frame, such as the downshift frame. The field of view includes a top sub-part for the phase shift frame and a lower sub-part that is not the top sub-part. The lower sub-part may be divided into one sub-part for compounding (e.g., a middle band) and a lower sub-part for just the downshift frame. Other bands and relative contributions within the bands may be provided.

For any bands or depths associated with compounding or contribution from both frames, the relative contribution may be predetermined. For example, a simple average or a weighted average with any change function over depth is used. Alternatively, the weights (e.g., relative contribution) are calculated from the data, such as from a difference between noise and the harmonic data. The description below for FIG. 5 shows one example.

The depth for adjusting the relative contribution is determined. The transition depth for the combination is determined as function of a comparison of the ultrasound data to noise. Either frame of data may be used. For example, the phase shift frame of data is used to determine the depth. The second harmonic of the phase shift frame is the primary or desired source of information if available. A noise frame is acquired, such as the noise frame used in act 54 of FIG. 2. For each band or depth, the noise signal is compared to the ultrasound data. In one embodiment, the bottom depth of each band is used for the comparison. From the bottom depth for each band, the noise signal is compared against the phase inversion signal. The comparison begins for the near field band. The first band without a significant statistical difference indicates the transition. The transition is a top of the band without a significant statistical difference or the bottom of the deepest band with a statistical difference.

Significant statistical difference may be an empirically determined threshold. Any statistic may be used. For example, the mean and variance are used. The statistic is calculated for the ultrasound data and for the noise. The statistical values are compared, such as differenced. The difference resulting from the comparison is used to locate the penetration depth. The penetration depth or a depth based on the penetration depth (e.g., 1 cm shallower than the detected penetration depth) indicates the transition to the downshifted frame.

Other techniques may be used for setting the transition depth. For example, the signal-to-noise ratio is used. In one embodiment, the transition is determined as part of creating the downshifted frame of data. In other embodiments, the transition is determined separately from creating the downshifted frame of data. Rather than calculating based on the currently acquired data for a given patient and/or scan, the transition depth may be predetermined or user selected.

In act 42, an image is generated. The transition depth defines the frames to be used for various depths. By combining the frames based on the transition depth, the resulting frame formed from data of multiple frequencies or types of harmonic imaging is created. This combination frame may be mapped to display values. An image is generated from the combined frame. For example, a tissue harmonic image having a band of second harmonic and a band of combined second harmonic and downshifted harmonic is generated. As another example, a tissue harmonic image having a near field representing tissue response at the second harmonic using phase shift and a far field representing tissue response based on downshifting is generated. The image may include more than two bands, such as a near field from the phase shift harmonic imaging, a far field from downshift harmonic imaging, and a middle field from a combination of phase shift and downshift.

FIG. 5 shows one embodiment of setting weights for frequency compounding in act 36 of FIG. 1. For frequency compounding, frames of data associated with different frequencies and/or different harmonic processing are blended. For example, the blending occurs in a band around, adjacent to, or after the transition point detected in act 40 of FIG. 4. The blending may occur for any sub-parts of the field of view where the downshift frame is used. As another example, the blending occurs for the entire field of view.

The weights are set or calibrated automatically. Alternatively, predetermined or user selected weights or a semi-automatic setting is used.

In acts 30 and 32, the frames of ultrasound data at the harmonic frequencies are acquired. For example, the phase shift frame is acquired using phase inversion second harmonic imaging, and a downshift frame of data is acquired as discussed for FIG. 2.

In act 46, the weights are calculated. The weights used for the weighted averaging in frequency compounding are set as a function of depth. Different weighting and relative contributions are provided for different depths. The weighting may be set for each band, such as determining weighting for each of eight bands. The weights are the same or different in different bands. For example, a plurality of bands closest to the transducer or in the near field may have the same or similar weights. For the far field, one or more bands have unique or different weights than used in other bands. Alternatively, the weighting is set by depth or the bands correspond to a single depth.

The weights are calculated to reduce over contribution of noise, such as avoiding heavy weighting of phase shift frame at depths beyond a penetration depth, and to provide a consistent look for the resulting image. By blending the phase shift frame with the downshift frame even near or beyond the penetration depth, a similar look may result.

In one embodiment, the weights are calculated as a function of a difference of the ultrasound data at the harmonic with noise. For each band, the frequency compounding weight of the phase shift frame is calculated. The information at one harmonic, such as the second harmonic is desired and the downshift is a mechanism to be close to the second harmonic but with better penetration.

The signal-to-noise ratio is used for setting the weights. The phase shift information or information at the second harmonic is compared to the noise. The weight is calculated according the deviation of the phase shift frame from the noise frame. The weight for the downshift frame is the difference of the weight for the phase shift frame from one. The weights for the different frames add to unity or one, but may add to other values. Where more than two frames are combined for a given spatial location, such as where steered spatial and frequency compounding are used, the function accounts for the number of frames.

For differencing from the noise, noise information is acquired, such as discussed above for act 54. The noise information and the phase shift information are used to determine the difference. For example, a square root of a square of a mean difference over covariance is calculated as the difference. Other examples include an absolute variance, ratio of mean to standard deviation, absolute difference, difference between minimum and maximum, statistical distribution difference, covariance, mutual information difference, or entropy difference.

Where the difference is greater or above a threshold, the weighting is greater for the phase shift information. Where the difference is lesser or below the threshold, the weighting is greater for the downshift information. Any mapping function of relative weights based on the difference of the harmonic information from noise may be used. The function is linear or non-linear.

In act 48, the frames are compounded. Weighted averaging is performed. For each location, the weights calculated for the corresponding depth are selected. The data for the location for each frame is multiplied by the corresponding weight. Data for different depths may be weighted the same or differently. The results from the different weighted frames are added. By applying the weights to the phase shift frame and the downshift frame and then blending (e.g., adding), frequency compounding is provided. The blending creates a consistent look over the range of depths while allowing for greater penetration than using just one harmonic.

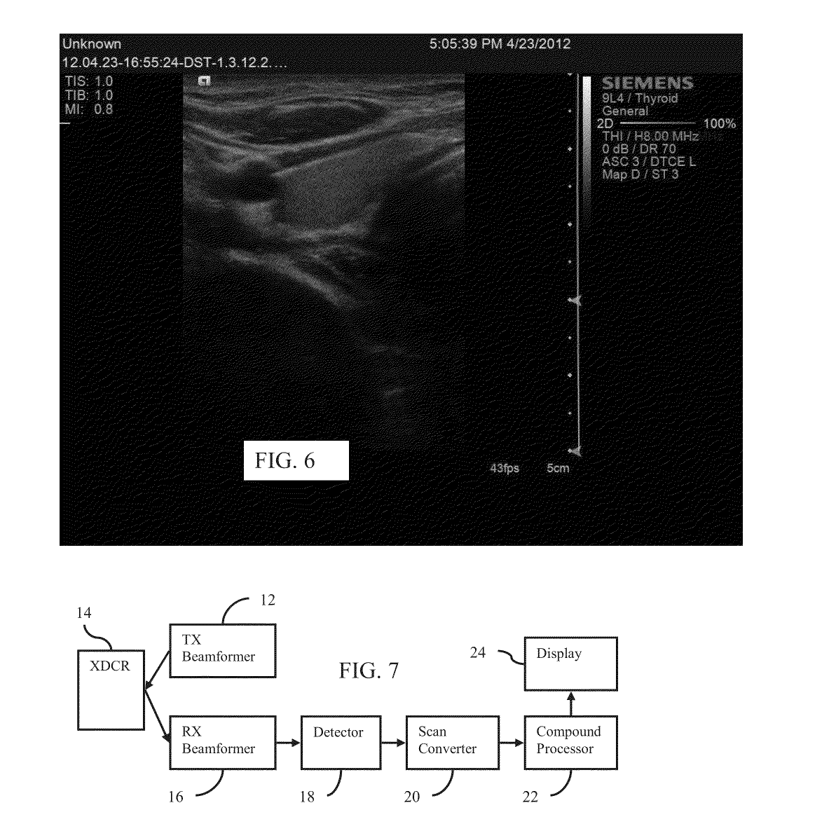

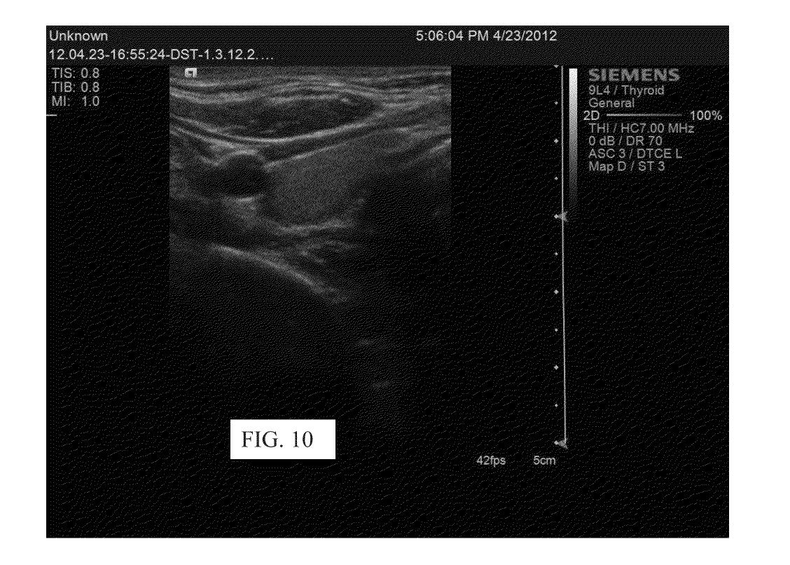

FIG. 6 shows an example image generated from a frame of data combined from a phase inversion frame and a downshift frame. The downshift frame is generated dynamically with continuous downshifting. The transition depth is determined based on the penetration of the phase inversion frame relative to noise. As shown in FIG. 8, the penetration is at about 4 cm. The weights applied to the combination are calculated based on or proportional to the difference of the phase inversion data from the noise. The image has better penetration and look-and-feel than the simple down-shift image in FIG. 9.

FIGS. 6, 8, 9, and 10 also include steered spatial compounding to further improve image quality. Furthermore, the weights between the downshift image and phase inversion image and between the steered spatial compounding angles may be dynamically adjusted based on the signal difference between the downshift image and phase inversion image, or between the steered spatial compounding angles.

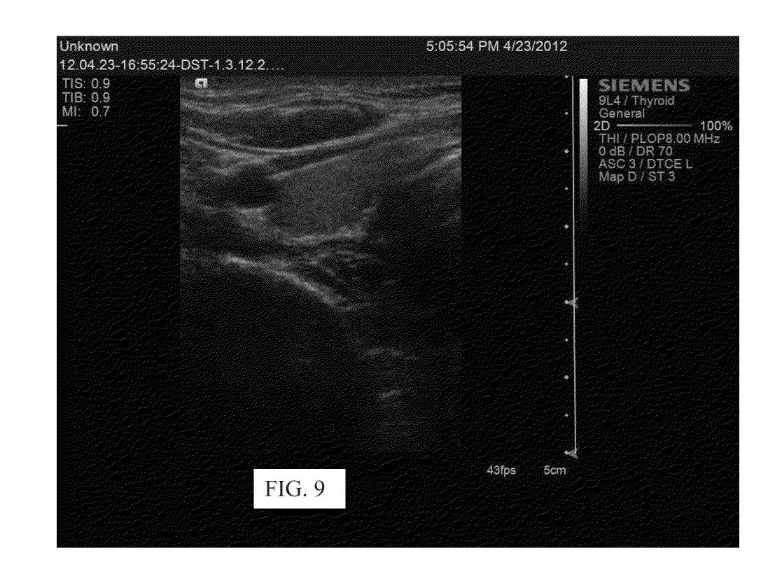

FIG. 7 shows a system 10 for frequency distribution in harmonic ultrasound imaging. The system 10 is a medical diagnostic ultrasound system. In alternative embodiments, all or part of the system 10 is a workstation or computer for processing or displaying medical images.

The system 10 includes a transmit beamformer 12, a transducer 14, a receive beamformer 16, a detector 18, a scan converter 20, a compound processor 22, and a display 24. Different, fewer or additional components may be provided. For example, an offline workstation implements the compound processor 22 and display 24 without the additional ultrasound acquisition components.

The transducer 14 comprises an one- or multi-dimensional array of piezoelectric, ceramic, or microelectromechanical elements. In one embodiment, the transducer 14 is a one-dimensional array of elements for use as Vector®, linear, sector, curved linear, or other scan format now known or later developed. The array of elements has a wavelength, half wavelength, or other sampling frequency. The transducer 14 is adapted for use external to or use within the patient, such as a handheld probe, a cardiac catheter probe, or an endocavity probe. Multiple spatially distributed transducers or even scanning systems may be employed.

The transmit and receive beamformers 12, 16 operate as a beamformer. As used herein, “beamformer” includes either one or both of transmit and receive beamformers 12, 16. The beamformer is operable to acquire frames of data responsive to different frequencies. Different scanning may be performed. For example, the beamformer transmits pulses with different relative phase and sums the received signals for phase shift harmonic. As another example, the beamformer includes a filter with a programmable band pass that filters received signals at programmable harmonic frequencies.

The transmit beamformer 12 is one or more waveform generators for generating a plurality of waveforms to be applied to the various elements of the transducer 14. The waveforms are at a fundamental frequency, such as 1-4 MHz. By applying relative delays and apodizations to each of the waveforms during a transmit event, a scan line direction and origin from the face of the transducer 14 is controlled. The delays are applied by timing generation of the waveforms or by separate delay components. The apodization is provided by controlling the amplitude of the generated waveforms or by separate amplifiers. To scan a region of a patient, acoustic energy is transmitted sequentially along each of a plurality of scan lines. In alternative embodiments, acoustic energy is transmitted along two or more scan lines simultaneously or along a plane or volume during a single transmit event. The waveforms are generated at any given phase, such as 0 and 180 degrees. The transmit beamformer 12 may generate the waveforms with the desired phase or may using a phase rotator.

The receive beamformer 16 comprises delays and amplifiers for each of the elements in the receive aperture. The receive signals from the elements are relatively delayed and apodized to provide scan line focusing similar to the transmit beamformer 12, but may be focused along scan lines different than the respective transmit scan line. The delayed and apodized signals are summed with a digital or analog adder to generate samples or signals representing spatial locations along the scan line. Using dynamic focusing, the delays and apodizations applied during a given receive event or for a single scan line are changed as a function of time. Signals representing a single scan line are obtained in one receive event, but signals for two or more scan lines may be obtained in a single receive event. A component frame of data is acquired by scanning over a complete pattern with the beamformer. In alternative embodiments, a Fourier transform or other processing is used to form a component frame of data by receiving in response to a single transmit.

The receive beamformer 16 includes a filter and/or a further summer for isolating information at a harmonic frequency, such as a 2-8 MHz frequency. The isolation may be relative rather than absolute, such as reducing signal from outside a desired band by 6, 10, or more dB. The filter may be a programmable filter or a bank of filters with different pass bands. A memory may be used to apply different filtering to the same signals. Alternatively, additional transmissions and receptions are performed to filter at different pass bands. A mixer may be used with a fixed or programmable low pass filter for isolating at a desired frequency. The summer may be used with a buffer to combine signals received in response to transmissions at different phases.

The detector 18 comprises a B-mode detector, Doppler detector or other detector. The detector 18 detects intensity, velocity, energy, variance or other characteristic of the signals for each spatial location in the component frame of data. The Doppler detector, corresponding corner turning memory and/or clutter filter may be used for isolating information at the harmonics instead of the receive beamformer 16. In other embodiments, the detector 18 is a harmonic tissue response detector.

The scan converter 20 comprises a processor, filter, application specific integrated circuit or other analog or digital device for formatting the detected data from a scan line format to a display or Cartesian coordinate format. The scan converter 20 outputs each frame of data in a display format.

The compound processor 22 comprises one or more memories, processors, control processors, digital signal processors, application specific integrated circuits, multiplexers, multipliers, adders, lookup tables and combinations thereof. In one embodiment, the compound processor 22 comprises a personal computer, motherboard, separate circuit board or other processor added to an ultrasound system for image processing using transfers of data to and from the ultrasound image generation pipeline or processing path (i.e. receive beamformer 16, detector 18, scan converter 20 and display 24). In other embodiments, the compound processor 22 is part of the image generation pipeline.

The compound processor 22 is configured by hardware and/or software. The compound processor 22 is configured to frequency compound frames of data. The compounded frames are of different harmonic frequencies. Different types of processing may be used for the frames to be compounded. The processor 22 may adaptively determine the weights used for compounding. The weights vary based on the signal-to-noise ratio or difference in characteristic of harmonic information from noise. The processor 22 may adaptively create a downshifted frame of harmonic data. For example, the processor 22 identifies penetration depth and adjusts the harmonic frequency used for different depths. The processor 22 may determine a transition depth from one type of harmonic imaging to another type of harmonic imaging.

The compound processor 22 is operable to combine detected and scan converted data. In alternative embodiments, the compound processor 22 is positioned between the detector 18 and scan converter 20 for combining detected but not scan converted data, positioned prior to a log compressor of the detector 18 for combining non-compressed information or positioned prior to the detector 18. Any of various embodiments for combining multiple data representing the same region or combining component frames of data may be used.

In one embodiment, the compound processor 22 includes an image display plane or memory for each of the component frames (e.g., frames with different harmonic frequencies), such as two display planes. Each display plane has foreground and background pages for allowing simultaneous writing to memory while reading out from memory, but other memory structures may be provided. The memory stores information for each spatial location, such as harmonic mode data. A filter responsive to different multiplier coefficients combines the component frames using different functions based on the contribution. A lookup table provides the different weighting coefficients to the multipliers. Different coefficients may also be provided for combining different numbers of component frames.

The instructions for implementing the processes, methods and/or techniques discussed above are provided on non-transitory computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system.

The display 24 is a CRT, monitor, flat screen, LCD, projection or other display for displaying the frequency compounded ultrasound image. Rather than compounding by the processor 22, the display plane memories may be used. During the display refresh, the component frames are read, weighted, summed and thresholded to generate the image on the display 24 where display plane memories are used for each component frame of data. The resulting frame of data is a frequency compound image responsive to component frames of data. Different locations have values from different component frames or from multiple or all of the component frames. The compound image is updated in real-time as subsequent frames of ultrasound data at the harmonic frequencies are available.

The display 24 is operable to display a compound image responsive to the component frames of data. The compound image reduces speckle while maintaining or increasing penetration depth with a similar look through the various depths. The combined frame of data is displayed as the compound image.

Other alternative embodiments include use for compounding three or four-dimensional images. Component frames of data are acquired with different lateral as well as elevation steering angles.

While the invention has been described above by reference to various embodiments, it should be understood that many changes and modifications can be made without departing from the scope of the invention. While the description herein provides examples of steered spatial compounding, other compounding, such as temporal or frequency compounding, may alternatively or additionally be used. It is therefore intended that the foregoing detailed description be regarded as illustrative rather than limiting, and that it be understood that it is the following claims, including all equivalents, that are intended to define the spirit and scope of this invention.

I (we) claim:

1. A method for frequency distribution in harmonic ultrasound imaging, the method comprising:
generating, with transmissions at different phases, a first frame of ultrasound data representing response of a region of a patient at one harmonic frequency;
generating, with filtering at different pass bands, a second frame of ultrasound data at a plurality of different harmonic frequencies as a function of depth;
combining the ultrasound data of the first frame with the ultrasound data of the second frame into a third frame, a relative contribution of the ultrasound data of the first frame to the ultrasound data of the second frame being a function of depth such that the contribution in a near field is greater from the first frame and the contribution in a far field is greater from the second frame; and
generating an image of the region with the combined ultrasound data of the third frame.

2. The method of claim 1 wherein generating with the transmissions at the different phases comprises phase inversion harmonic imaging at a second harmonic.

3. The method of claim 1 wherein generating with the filtering at the different pass bands comprises downshift harmonic imaging.

4. The method of claim 1 wherein generating with the filtering at the different pass bands comprises filtering with a pass band at a first frequency in a near field and downshifting the pass band by increasing amounts from the first frequency as a function of increasing depth.

5. The method of claim 4 further comprising:
calculating depths for the downshifting by the increasing amounts as a function of a comparison of the ultrasound data at a current pass band to noise.

6. The method of claim 1 wherein combining comprises determining a depth for adjusting the relative contribution as a function of a comparison of the first ultrasound data to noise.

7. The method of claim 1 wherein generating the first frame comprises generating the first frame for the region, the region being a field of view, wherein generating the second frame comprises generating the second frame for a first sub-part of the region, wherein combining comprises combining with different relative contributions over a second sub-part of the region, the second sub-part comprising a band of depths within but less than all of the first sub-part, a third sub-part comprising a remaining portion of the first sub-part outside the second sub-part, the third sub-part being formed from the second and not the first ultrasound data, and a fourth sub-part of the region being formed from the first and not the second ultrasound data, and wherein generating the image of the region comprises generating an image of the second, third and fourth sub-parts.

8. The method of claim 1 wherein generating the image comprises generating a tissue harmonic image having a band of second harmonic and a band of combined second harmonic and downshifted harmonic.

9. The method of claim 1 wherein combining comprises calculating the relative contribution as a function of a difference between noise and the first ultrasound data.

10. In a non-transitory computer readable storage medium having stored therein data representing instructions executable by a programmed processor for frequency distribution in harmonic ultrasound imaging, the storage medium comprising instructions for:
detecting a first penetration depth of harmonic imaging at a first harmonic frequency;
shifting to a second harmonic frequency lower than the first harmonic frequency at the first penetration depth;
detecting a second penetration depth of the harmonic imaging at the second harmonic frequency; and
shifting to a third harmonic frequency lower than the first harmonic frequency at the second penetration depth.

11. The non-transitory computer readable storage medium of claim 10 wherein detecting the first and second penetration depths comprises detecting a signal-to-noise ratio below a threshold.

12. The non-transitory computer readable storage medium of claim 10 wherein detecting the first and second penetration depths comprises:
acquiring noise data; and
comparing the noise data to ultrasound data for the first and second harmonic frequencies.

13. The non-transitory computer readable storage medium of claim 12 wherein comparing comprises comparing a mean and variance of the ultrasound data and the noise data.

14. The non-transitory computer readable storage medium of claim 12 wherein comparing comprises comparing a histogram of the noise data and the ultrasound data.

15. The non-transitory computer readable storage medium of claim 10 wherein detecting for the first harmonic frequency comprises detecting for a second order of a fundamental frequency and wherein detecting for the second harmonic frequency comprises detecting for a fractional harmonic less than the second order of the fundamental frequency.

16. The non-transitory computer readable storage medium of claim 10 wherein a downshifted frame of data is generated with the detecting and shifting;
further comprising:
generating a phase shift frame of data;
detecting a third penetration depth of the phase shift frame of data; and
combining the phase shift frame of data with the downshifted frame of data where the phase shift frame of data predominately contributes in a near field and the downshifted frame of data has a predominately contributes in a far field, the third penetration depth separating the near field from the far field.

17. The non-transitory computer readable storage medium of claim 16 further comprising:
blending the phase shift frame of data with the downshift frame of data in a band of depths; and
setting weights for the blending as a function of a signal-to-noise ratio for the phase shift frame of data.

18. In a non-transitory computer readable storage medium having stored therein data representing instructions executable by a programmed processor for frequency distribution in harmonic ultrasound imaging, the storage medium comprising instructions for:
acquiring first and second sets of harmonic data, the first set of harmonic data being different than the second set of harmonic data;
compounding the harmonic data of the first set with the harmonic data of the second set, the compounding being a weighted average; and
calculating weights of the weighted average as a function of a difference of the harmonic data of the first set with noise.

19. The non-transitory computer readable storage medium of claim 18 wherein acquiring comprises acquiring the first set as phase inversion data representing response at a second order of a fundamental frequency and acquiring the second set as filtered data representing response at fractional orders of the fundamental frequency.

20. The non-transitory computer readable storage medium of claim 18 wherein calculating the weights comprises calculating the weights as function of the difference at different depths, the weights being different for the different depths.

21. The non-transitory computer readable storage medium of claim 18 wherein calculating the weights comprises calculating a square root of a square of a mean difference over covariance as the difference.

22. The non-transitory computer readable storage medium of claim 18 wherein the difference is a function of entropy, mutual information distance, ratio of mean to standard deviation, absolute difference, difference between minimum and maximum, statistical distribution difference, or combinations thereof.

23. The non-transitory computer readable storage medium of claim 18 wherein calculating the weights comprises calculating as a function of steering angles.
